(19) United States
(12) Patent Application Publication
(10) Pub. No.: US 2003/0109016 A1
(43) Pub. Date: Jun. 12, 2003

(54) TSLL1 gene

(75) Inventors: Yoshinori Murakami, Tokyo (JP); Sachio Nomura, Saitama (JP)

Correspondence Address: SUGHRUE MION, PLLC, 2100 PENNSYLVANIA AVENUE, N.W., WASHINGTON, DC, 20037, US

(73) Assignee: PRESIDENT, NATIONAL CANCER CENTER and BML, INC.

(21) Appl. No.: 10/230,335

(22) Filed: Aug. 29, 2002

(30) Foreign Application Priority Data
Oct. 11, 2001 (JP) 2001-313966

(51) Int. Cl. C12N 9/00; C07H 21/04; C12N 1/21; C12N 5/06; C07K 16/40

(52) U.S. Cl. 435/183; 435/252.3; 435/325; 435/320.1; 536/23.2; 530/388.25

(57) ABSTRACT

A tumor suppressor gene (TSLL1 gene) having a nucleotide sequence encoding an amino acid sequence of SEQ ID NO: 1. The gene is useful in the prevention and treatment of cancers. Proteins, vectors, transformants and antibodies related to the gene are also disclosed.

### BACKGROUND OF THE INVENTION

1. Field of the Invention

The present invention relates to a specific type of gene, and more particularly, to a tumor suppressor gene, as well as to proteins, vectors, transformants, and antibodies related to the tumor suppressor gene.

2. Description of the Related Art

As cancer research has progressed, tumor suppressor genes, which are known to play a role of suppressing the onset of cancers, have attracted keen interest. Thus far, a great number of tumor suppressor genes have been identified.

To date, the following tumor suppressor genes have been reported, among others: the RB gene for retinoblastoma, the NF1 and NF2 genes for neurofibromatosis, the APC gene for familial polyposis coli, the WT1 gene for Wilms' tumor, the MEN1 gene for multiple endocrine neoplasia type 1, the p53 gene for Li-Fraumeni syndrome, the VHL gene for von Hippel-Lindau syndrome, the BRCA1 and BRCA2 genes for familial breast cancer, the p16 gene for familial melanoma, the TSC1 and TSC2 genes for nodular sclerosis (sarcoma, brain tumor), and the PTCH gene for basal cell nevus syndrome.

Tumor suppressor genes are known to prevent carcinogenesis of cells primarily through suppression of cell proliferation. Also, mismatch repair genes and other cell-adhesion-related genes are also thought to participate in suppression of carcinogenesis.

Tumor suppressor genes are very useful in, for example, diagnoses of carcinoma in precritical stages, qualitative diagnoses of carcinoma, prediction of prognosis of cancer therapy, prediction of possible metastasis of carcinoma, forecast of sensitivity of carcinoma to chemical therapy or radiotherapy, and gene therapy. Thus, studies on tumor suppressor genes are being performed energetically around the clock, so as to discover as many tumor suppressor genes as possible.

### SUMMARY OF THE INVENTION

The present inventors identified a tumor suppressor gene, TSLC1, in non-small cell lung cancers. The TSLC1 gene is located on the long arm of chromosome 11 and very often exhibits loss of heterozygosity. The tumor suppressor gene, TSLC1, identified with reference to suppression of tumorigenicity of lung adenocarcinoma cells A549 as an indicator exhibits tumor suppressor activity for numerous cells of malignant carcinoma, making the tumor suppressor gene TSLC1 a novel candidate target of cancer therapy.

In relation to the mentioned tumor suppressor gene TSLC1, an object of the present invention is to identify another tumor suppressor gene having a structural or functional relationship to the gene TSLC1.

Another object of the invention is to provide novel means useful for prevention or treatment of cancers.

With an aim toward attainment of the above objects, the present inventors have extended their studies regarding the tumor suppressor gene TSLC1, and have identified a gene exhibiting novel tumor suppressor functions, the gene being different from TSLC1 but homologous thereto, locating on chromosome 1, and containing a nucleotide sequence encoding the amino acid sequence represented by SEQ ID NO: 1, thus completing the present invention.

Accordingly, the present invention provides a gene comprising a nucleotide sequence that encodes an amino acid sequence of SEQ ID NO: 1 (hereinafter may be referred to as the present gene or TSLL1 gene).

### BRIEF DESCRIPTION OF THE DRAWINGS

Various other objects, features and many of the attendant advantages of the present invention will be readily appreciated as the same becomes better understood by reference to the following detailed description of the preferred embodiments when considered in connection with the accompanying drawings, in which:

FIG. 1 shows a nucleotide sequence of TSLL1 cDNA and an amino acid sequence deduced therefrom;

FIG. 2 schematically shows structural similarity among amino acid sequences which are encoded by the present gene or other genes related to the present gene;

FIG. 3 schematically shows the present gene and the human TSLC1 gene;

FIG. 4 shows the locus of the present gene on a chromosome;

FIG. 5 shows the results of a Northern blotting analysis of the present gene in adult and fetal tissue samples;

FIG. 6 shows the results of a Northern blotting analysis of the present gene in a cell line of human glioma; and

FIG. 7 shows successful detection with specificity of TSLL1 protein, attained by use of anti-TSLL1 polyclonal antibody AC2.

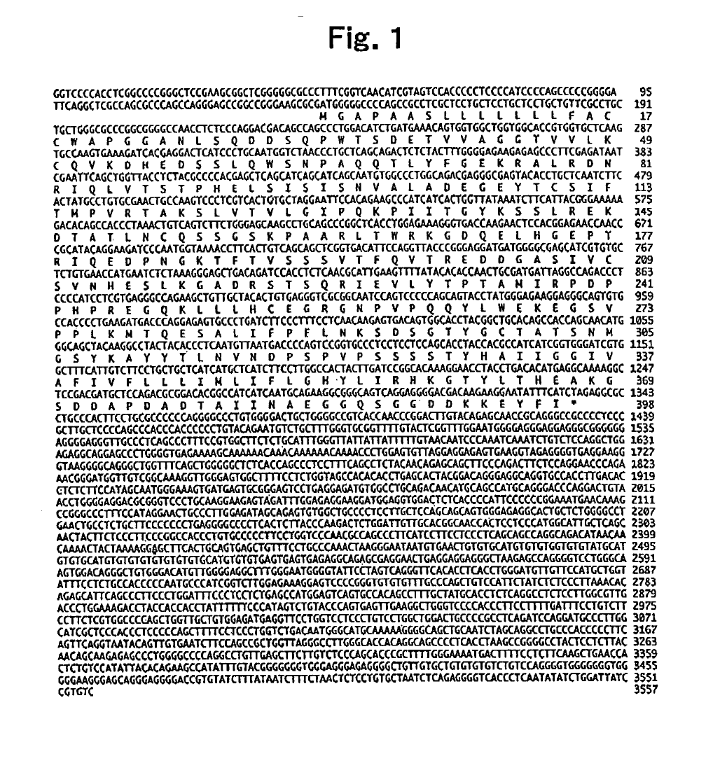

### DESCRIPTION OF THE INVENTION AND PREFERRED EMBODIMENTS

The manner in which the present inventors have discovered the present gene will be disclosed in the “EXAMPLES” section in the present specification.

In the specification, both the amino acid three-letter code and the one-letter code are employed. That is, as used herein, alanine may be expressed by Ala (three-letter code) or A (one-letter code), valine by Val (V), leucine by Leu (L), isoleucine by Ile (I), proline by Pro (P), phenylalanine by Phe (F), tryptophan by Trp (W), methionine by Met (M), glycine by Gly (G), serine by Ser (S), threonine by Thr (T), cysteine by Cys (C), glutamine by Gln (Q), asparagine by Asn (N), tyrosine by Tyr (Y), lysine by Lys (L), arginine by Arg (R), histidine by His (H), aspartic acid by Asp (D), and glutamic acid by Glu (E).

Modes for carrying out the present invention will next be described.

A. Description of the Present Gene

As described above, the present gene contains a nucleotide sequence that encodes the amino acid sequence represented by SEQ ID NO: 1.

The present gene has a length of about 30 kb and includes nine exons; i.e., exon 1, seven subsequent exons each having a relatively small size of 91 to 171 bp, and a large exon 9. Exon 1 includes an initiation codon for methionine, which is located downstream the Kozak's consensus sequence. In the upstream region of the present gene are rich in guanine content and cytosine content, and a CpG island region of 754 bp is identified.

On the basis of the amino acid sequence of SEQ ID NO: 1 predicted from the sequence of the present gene, the present gene is speculated to code for a transmembrane domain, a short cytoplasmic domain, and three immunoglobulin-like C2-type loops containing potential N-glycosylation sites. In the cytoplasmic domain, the amino acid sequence encoded by the present gene exhibits 76% homology with that encoded by tumor suppressor gene TSLC1. For the entire sequence, the amino acid sequence encoded by the present gene exhibits 37% homology with that encoded by tumor suppressor gene TSLC1. Therefore, the protein encoded by the present gene is considered to belong to an immunoglobulin superfamily.

The present gene is located on chromosome 1 (1q21.2-22), and is specifically expressed in the human brain.

The present gene is deeply involved in development, as a minimum, of carcinoma which originates from brain or nerve cells. In particular, the present gene functions to suppress development of carcinoma in the brain or nerve cells.

B. Production of the Present Gene

The present gene can be produced through, for example, PCR or a similar gene amplification technique, in which cDNA prepared from human brain-tissue-derived poly(A)⁺RNA is employed as a template.

Specifically, a cDNA capable of encoding TSLL1 is prepared through a conventional method (for example, a method in which a reverse transcriptase and poly(A)⁺RNA serving as a template are employed), and the thus-prepared cDNA is amplified through a gene amplification technique such as PCR, to thereby prepare the present gene.

Primers needed for PCR amplification may be those specific to the TSLL1 gene.

Specifically, each of the primers to be employed may be a DNA fragment containing a sequence identical with the sequence of the 5′ or 3′ terminal portion of the coding region of the present gene, the sequence of which is determined in a manner described hereinafter. By use of such primers, the gene can be amplified to a large amount, and the thus-amplified gene may be provided for use.

No limitations are imposed on the nucleotide sequence of the primers employed for gene amplification, and any sequences may be employed in accordance with the gene amplification technique employed. Generally, primers each having a complementary nucleotide sequence to the 5′ or 3′ terminal portion of the region to be amplified are employed. By use of such primers, the portion corresponding to the present gene, which is located between the primers, can be amplified. Moreover, no limitations are imposed on the length of the nucleotides contained in a primer for gene amplification, and the length of the nucleotides is generally about 17 to about 35, preferably about 20 to about 30. When the length of the nucleotides contained in the primer is excessively small, accuracy of hybridization to the target region is low, whereas when the number of the bases is excessively large, cost for gene amplification increases and handling of the primer tends to become cumbersome. Preferably, the primer is complementary as strict as possible to the hybridization region serving as a template, in order to attain ensured hybridization. In particular, the primer sequence on the 3′ terminus is preferably highly complementary to the template sequence.

Examples of the primers for gene amplification include, but are not limited to, the following nucleotide sequences.

5′-CCTTTCGGTCAACATCGTAGTCCACCCCCT-3′

(SEQ ID NO: 3)

5′-TACAAGTCCGGGTTGGTGACGGCCCCAGCA-3′

(SEQ ID NO: 4)

These specific primers for gene amplification are generally prepared through chemical synthesis. In accordance with needs, the primers may include an appropriate nucleotide sequence containing a site which can be recognized by a restriction endonuclease.

By use of the thus-obtained amplified gene product, DNA fragments containing TSLL1 gene can be concentrated through, for example, the following process: A polynucleotide having a nucleotide sequence which is specific to TSLL1 gene is labeled; the labeled polynucleotide serving as a probe is hybridized with the amplified product; and cDNA fragments containing TSLL1 gene are selected with the aid of the label.

No particular limitations are imposed on the labeling, so long as the above-described selection can be carried out, and, naturally, labeling and selection are preferably conducted through as simple means as possible. From this standpoint, use of a method in which, for example, the cDNA fragments are labeled with biotin and the labeled cDNA fragments are adsorbed onto streptavidin is advantageous.

In the above-described manner, desired cDNA of the present gene can be obtained.

The thus-prepared present gene is inserted in an appropriate gene transfer vector, and the resultant vector is introduced into a host cell suitable for the gene transfer vector, whereby a transformant in which the present gene has been introduced can be obtained. Whether or not the cDNA fragment has been integrated in the gene transfer vector can be determined through, for example, selection of color developed by the activity of lac Z gene carried by the vector.

The present gene may also be transferred into a vector through use of a commercial kit for preparing a gene library.

A clone carrying the present gene may be obtained through the following steps: preparing a polynucleotide having a nucleotide sequence specific to the TSLL1 gene; labeling the polynucleotide; hybridizing the labeled polynucleotide serving as a labeled probe with replicas of respective clones; and isolating clones which have hybridized the polynucleotide serving as a labeled probe. The thus-isolated clones can be used as clones carrying the present gene, TSLL1.

The nucleotide sequence of the thus-prepared present gene can be identified through a conventional method.

For example, there may be employed the Maxam-Gilbert method (Maxam, A. M., and Gilbert, W., Proc. Natl. Acad. Sci. U.S.A., 74, 560 (1977)), the dideoxy method (Sanger, F., et al., Proc. Natl. Acad. Sci. U.S.A., 74, 5463 (1977)), the genomic sequence method (Church, G. M. and Gilbert, W., Proc. Natl. Acad. Sci. U.S.A., 81, 1991 (1984)), the multiplex method (Church, G. M. and Kieffer-Higgins, S., Science, 240, 185 (1988)), or the cycle sequencing method (Murray, V., Nucleic Acids Res., 17, 8889 (1989)).

As a matter of course, the nucleotide sequence may be determined through use of an automatic analyzer for nucleotide sequences, designed on the basis of the principles of the above methods.

Fragments of the present gene may be chemically synthesized through a method known per se, such as the phosphite-triester method (Ikehara, M., et al., Proc. Natl. Acad. Sci. U.S.A., 81, 5956 (1984)). Moreover, fragments of the present gene may be synthesized by use of a DNA synthesizer to which such a chemical synthesis method is applied.

The thus-produced gene of the present invention may be modified through deletion, substitution, insertion, or addition of a portion of the nucleotide sequence of the present gene, to thereby produce a modified gene. The present inventors have well recognized the existence of such modified genes, and thus such modified genes fall within the scope of the present invention, so long as the modified genes have about 90% or more homology to the present gene in a non-modified form even in the case in which a large portion of the nucleotide sequence of the present gene is deleted, substituted, inserted, or added.

A modified gene of interest can be produced through a method known per se, such as so-called site-specific mutagenesis (Mark, D. F., et al., Proc. Natl. Acad. Sci. U.S.A., 81, 5662 (1984)).

The genes encompassed by the scope of the present invention are such genes that can be hybridized with DNA having a nucleotide sequence represented by SEQ ID NO: 2 (described hereinafter) under stringent conditions, and that encode TSLL1 protein exhibiting biological activity substantially identical to that of TSLL1 protein having an amino acid sequence of SEQ ID NO: 1 (described hereinafter). The term “stringent conditions” is used to refer to conditions which tend to inhibit hybridization between DNA strands in the reaction system. The level of stringency depends on, for example, temperature of the system (the higher the temperature of the system, the harder hybridization is to be attained), salt concentration (the lower the salt concentration, the harder hybridization is to be attained) and concentration of the denaturing agent such as formamide (the higher the concentration of the denaturing agent, the harder hybridization is to be attained).

As used herein, “substantially identical” means that the biological activity is qualitatively and/or quantitatively identical with that of TSLL1 used for comparison.

C. Utility of the Present Gene

(1) Utility in Genetic Diagnosis

The present gene finds utility in gene diagnosis of cancers. Specifically, the following 1) and 2) can be realized.

1) The present gene acts to suppress brain or nerve cell tumors. Therefore, the gene enables study in terms of malignancy of a tumor biopsy specimen in the course of gene analysis in which at least the present gene is employed as a target gene to be analyzed, together with pathological diagnosis using staining profiles obtained through, for example, staining of the biopsy specimen with hematoxylin-eosin.

Specifically, among DNA molecules of a biopsy specimen, whether the present gene is found to have mutation or deletion would be employed as a risk determining factor for the malignancy of a tumor in the donor of the specimen. Among DNA molecules of the biopsy specimen, when the present gene is found to have neither mutation nor deletion, the present gene is considered to function as a tumor suppressor, and such a result may be connected to low level of malignancy. Conversely, if mutation or deletion is identified, the present gene is considered to not function properly, and such a result may be connected to high level of malignancy.

The aforementioned mutation or deletion may be employed as a risk determining factor for the prognosis of a subject. Specifically, since the present gene is speculated to be involved in cell adhesion or a similar phenomenon, the present gene is considered to function suppressively on invasion or metastasis of cancers. Therefore, mutation, methylation or deletion of the present gene may be employed as a risk determining factor concerning invasion or metastasis of a target tumor.

2) Detection of abnormality in the present gene before the onset of a tumor may be a useful risk determining factor with respect to the development of a brain or nerve tumor of a subject.

When analysis of a specimen from a subject reveals that the present gene has abnormality such as mutation, methylation or deletion, it is concluded that the present gene does not function properly as a tumor suppressor gene, and such a result may be employed as a risk determining factor for high incidence of the onset of tumors in the brain or nerves of a subject.

(2) Utility in Gene Therapy

Specifically, if the aforementioned gene diagnosis reveals that the present gene includes a mutation, methylation or deletion to the effect that the present gene fails to function properly, the present gene in its normal form is administered to a subject (including both a subject who has yet to develop a cancer of interest and a subject who has already developed a cancer), whereby suppression or therapy of the cancer is realized.

Briefly, the present gene (preferably, the gene is in the form in which a promotor, etc. is linked for expression of the gene) is incorporated into a gene transfer vector (for example, a retrovirus vector or an adenovirus vector) which can be used in gene therapy, and subsequently, the resultant vector is administered to a subject in need, to thereby restore the functions of the present gene in the subject. Thus, the present gene can be used to prevent the onset of a cancer or treat a cancer.

D. Production of TSLL1 Protein

A recombinant TSLL1 protein may be produced by use of the thus-present gene; i.e., the TSLL1 gene. Hereafter, the recombinant TSLL1 protein may be referred to simply as the TSLL1 protein. Unless otherwise specified, the TSLL1 protein encompasses TSLL1 proteins that can be obtained through transcription and translation of the aforementioned modified gene. Needless to say, such modified proteins exhibit biological activity substantially identical with that of unmodified TSLL1 protein.

The TSLL1 protein can be produced by use of the present gene in accordance with a conventional recombinant technique known per se.

Specifically, the present gene is inserted into a gene expression vector capable of expressing the present gene. The resultant recombinant vector is transferred to a host appropriately selected in consideration of the nature of the gene expression vector, whereby the cells are transformed. Subsequently, the transformants are cultured, to thereby produce the TSLL1 protein of interest.

The gene expression vectors to be used herein preferably have, within an upstream region of a gene to be expressed, an enhancer, a promotor, and translation initiation sequences, and, within a downstream region, a transcription termination sequence.

The expression system for the present gene is not limited to only a direct expression system, but may be a fusion protein expression system making use of, for example, a β-galactosidase gene, a gultathione-S-transferase gene, or a thioredoxin gene.

Examples of a gene expression vector whose host is E. coli include pQE, pGEX, pT7-7, pMAL, pTrxFus, pET, and pNT26CII. Examples of a gene expression vector whose host is Bacillus subtilis include pPL608, pNC3, pSM23, and pKH80.

Examples of a gene expression vector whose host is yeast include pGT5, pDB248X, pART1, pREP1, YEp13, YRp7, and YCp50.

Examples of a gene expression vector whose host is a mammalian cell or insect cell include p91023, pCDM8, pcDL-SRα296, pBCMGSNeo, pSV2dhfr, pSVdhfr, pAc373, pAcYM1, pRc/CMV, pREP4, and pcDNAI.

These gene expression vectors may be selected in accordance with the purpose of expression of the TSLL1 protein. For example, when the TSLL1 protein is desired to be expressed in large amounts, a gene expression vector which allows use of E. coli, Bacillus subtilis, or yeast as a host is preferably employed. On the other hand, if ensured expression of the TSLL1 protein—though in small amounts—is desired, a gene expression vector which allows use of a mammalian cell or insect cell as a host is preferably employed.

Although a gene expression vector may be selected from among existing ones as described above, as a matter of course, an appropriate gene expression vector may be created so as to meet the purpose of expression.

Such gene expression vectors also fall within the scope of the present invention.

The aforementioned gene expression vectors in which the present gene is inserted are transferred to host cells, and then the cells are transformed through a conventional method; for example, the calcium chloride method or electroporation in the case where the host is E. coli or Bacillus subtilis, or the calcium phosphate method, electroporation, or the liposome method in the case where the host cells are mammalian cells or insect cells.

The resultant transformed cells are cultured through a conventional method, to thereby yield the TSLL1 protein of interest (such transformants also fall within the scope of the present invention).

A culture medium is appropriately selected so as to meet the properties of the host. For example, when the host is E. coli, LB medium or TB medium may be used, and when the host is a mammalian cell, RPMI1640 medium may be used.

The TSLL1 protein can be isolated and purified from the resultant culture product through a conventional method. For example, it can be isolated and purified by use of any of a variety of treatment procedures making use of physical and/or chemical properties of the TSLL1 protein.

Specifically, isolation and purification of the protein may be performed through treatment making use of a protein precipitant, ultrafiltration, gel filtration, high-performance liquid chromatography, centrifugal separation, electrophoresis, affinity chromatography by use of a specific antibody, or dialysis. These are used singly or in combination.

In this way, the TSLL1 protein can be isolated and purified.

Use of the TSLL1 protein as an antigen facilitates production of an antibody that specifically recognizes TSLL1, and the resultant antibody can be used for, for example, diagnostic purposes. Also, a structural analog of TSLL1 protein may be produced on the basis of the TSLL1 protein, and a molecule which activates the functions of the TSLL1 protein may be isolated or synthesized, whereby such a molecule can be employed in the treatment of glioma. By causing TSLL1 protein to be expressed in cells, intracellular sites where the protein is localized may be determined, and other proteins which bind to and interact with the TSLL1 protein in cells can be identified. Thus, not only the TSLL1 protein per se but also the bound proteins find utility in elucidation of the pathway through which brain tumor, such as glioma, can be suppressed, and in identification and synthesis of substances that suppress development of such a tumor.

E. Production of Antibody Against TSLL1 Protein

The present invention is also directed to an antibody against the TSLL1 protein. The antibody will be also hereinafter referred to as the present antibody. When the present antibody is a polyclonal antibody, it will be also referred to as the present polyclonal antibody; and when the present antibody is a monoclonal antibody, it will be also referred to as the present monoclonal antibody.

The present polyclonal antibody can be produced by use of immune serum derived from animals which are immunized with TSLL1 protein serving as an immunogen.

In this context, no particular limitation is imposed on the TSLL1 protein that can be used as an immunogen. Not only a TSLL1 protein encoded by the present gene—the gene encompassing a TSLL1 gene in which a portion of the nucleotide sequence is modified—prepared by the foregoing method, but also fragments of the TSLL1 protein encoded by a partial fragment of the present gene, as well as partial peptides of the TSLL1 protein, the peptides being obtained through direct enzyme treatment of the TSLL1 protein or chemical synthesis, may be used as immunogens for producing the present polyclonal antibody. When a partial peptide derived from the TSLL1 protein is small, if necessary, a carrier protein is bound to the peptide, and the resultant peptide may be used as an immunogen.

Alternatively, the present polyclonal antibody can be prepared in the following manner: A cell strain derived from an animal which is of the same species and genealogy as the animal to be immunized is transformed with an expression vector which harbors a gene encoding the TSLL1 protein (the TSLL1 protein encompasses such modified TSSL1 proteins as described above) or a portion of the TSSL1 protein. Subsequently, the transformed cells are transplanted to the animal to be immunized, whereby the present polyclonal antibody can be produced. Specifically, the transformed cells continuously produce TSLL1 protein within the body of the animal to which the transformed cells have been transplanted. As a result, an antibody against the TSLL1 protein is produced, which may be used as the polyclonal antibody of the present invention (Nemoto, T., et al., Eur. J. Immunol., 25, 3001 (1995)).

Similar to the above-described case in which the transformed cells are transplanted to an animal, the present polyclonal antibody can also be produced by directly administering, to an animal, an expression vector for expressing the TSLL1 protein through, for example, intramuscular injection or subcutaneous injection, so as to elicit continuous production of the TSLL1 protein in the body of the animal (Raz, E., et al., Proc. Natl. Acad. Sci. U.S.A., 91, 9519 (1994)).

The present monoclonal antibody can be produced in a manner similar to that employed for the production of the present polyclonal antibody. Briefly, hybridomas between immune cells of an immunized animal and animal myeloma cells are created, and by use of the thus-created hybridomas, clones which produce antibodies recognizing the TSLL1 protein are selected and cultured, whereby the mentioned monoclonal antibody can be obtained.

No particular limitation is imposed on animals to be immunized. For example, mouse, rat, and other animals chosen from among a broad range of animals may be used. However, in producing a monoclonal antibody, the animals are preferably selected in consideration of compatibility of the animal with myeloma cells to be used in cell fusion.

Immunization may be performed through a conventional method, For example, the aforementioned immunogen is intravenously, intradermally, subcutaneously, or intrapertioneally injected to an animal to be immunized.

More specifically, the aforementioned immunogen, if necessary, together with a conventional adjuvant, is administered to the animal several times every two to 14 days by the aforementioned means, whereby immune serum for producing polyclonal antibodies, or immune cells for producing monoclonal antibodies—such as immunized spleen cells—can be obtained.

In the case of production of the monoclonal antibody, any of the following known myeloma cells may be used as a parent cell and fused with the immune cell: SP2/0-Ag14, P3-NS1-1-Ag4-1, MPC11-45, and 6.TG1.7 (all of which are derived from mouse); 210.RCY.Ag1.2.3 (derived from rat); and SKO-007 and GM15006TG-A12 (both are derived from human).

Cell fusion of the immune cells and the myeloma cells can be performed in accordance with a method known per se; for example, the method proposed by Kohler and Milstein (Kohler, G. and Milstein, C., Nature, 256, 495 (1975)).

Specifically, cell fusion may be performed as follows: In the presence of a fusogen such as polyethylene glycol (PEG) or Sendai virus (HVJ), immune cells and myeloma cells are cultured in a conventional culture medium in which an auxiliary agent such as dimethylsulfoxide has been added according to needs in order to enhance fusion efficiency, to thereby produce hybridomas.

In order to isolate a hybridoma of interest, the hybridomas are cultured in conventional selection medium; e.g., HAT (hypoxanthine, aminopterin, and thymidine) medium. That is, the hybridoma of interest can be isolated by culturing hybridomas for a certain period of time sufficient to kill cells other than the hybridoma of interest. Through a conventional limiting dilution technique, the resultant hybridoma can be subjected to screening for a monoclonal-antibody-producing strain of interest and cloning.

Screening for a monoclonal-antibody-producing strain of interest may be performed through a conventional method; for example, ELISA, plaque technique, spot test, agglutination test, Ouchterlony test, or RIA.

The resultant hybridoma that produces a monoclonal antibody which recognizes the TSLL1 protein can be subcultured in a conventional medium, and can be stored in liquid nitrogen for a long time (such a hybridoma falls within the scope of the present invention).

A monoclonal antibody of interest may be collected from a culture supernatant after the hybridoma is cultured by a customary method. Alternatively, the monoclonal antibody may be also collected by administering the hybridoma to an animal having compatibility with the hybridoma, to thereby induce multiplication of the hybridoma, and collecting the ascites from the animal.

Alternatively, the monoclonal antibody of interest may be also obtained as follows. Immune cells are cultured in vitro in the presence of the TSLL1 protein or a portion thereof, and after a certain period, hybridomas of the cultured immune cells and myeloma cells are prepared through the aforementioned cell fusion method. Subsequently, antibody-producing hybridomas are screened, to thereby obtain the monoclonal antibody of interest (Reading, C. L., J. Immunol. Meth., 53, 261 (1982); Pardue, R. L., et al., J. Cell Biol., 96, 1149 (1983)).

Alternatively, the monoclonal antibody of interest may be also produced by direct use of the present gene or a portion thereof as an immunogen, without use of the TSLL1 protein as an immunogen.

Specifically, an animal is directly immunized with the present gene (in this case, a recombinant gene expression vector which harbors the present gene may be used as an immunogen), and by use of immune cells or immune serum of the animal immunized with the present gene, monoclonal antibodies which recognize the TSLL1 protein with specificity can be produced.

The thus-obtained polyclonal and/or monoclonal antibodies may be purified by conventional means, such as salting out, gel filtration, or affinity chromatography.

The thus-obtained polyclonal and/or monoclonal antibodies have specific reactivity with the TSLL1 protein.

By use of an antibody against TSLL1, expression of TSLL1 protein in cells or tissue can be detected with specificity. Therefore, the antibody against TSLL1 can be used in diagnosis, differential diagnosis, and qualitative diagnosis of various pathological conditions of the brain or nerves, such as glioma.

### EXAMPLES

The present invention will next be described in more detail by way of examples.

Isolation and Sequencing of cDNA Clones

An amino acid sequence homologous with respect to 46 amino acid residues of the entire cytoplasmic domain of TSLC1 was determined by use of the advanced BLAST program (http://www.ncbi.nlm.nih.gov/BLAST/). A human adult brain-derived cDNA library, Lambda TriplEX, was purchased from Clontech. A probe used for screening of a cDNA library was a 1103 bp cDNA fragment obtained through PCR for TSLL1. The probe was obtained by use of the following primers.

Primer 1: 5′-ACAGCCAGCCCTGGACAT-3′ (SEQ ID NO: 5)

Primer 2: 5′-CTAGATGAAATATTCCTTCTTGT-3′ (SEQ ID NO: 6)

The DNA sequence was determined by means of a BigDye terminator cycle sequencing ready kit (Product of Perkin-Elmer) on an ABI 377 DNA auto-sequencer (Product of Applied Biosystems).

Firstly, two corresponding nucleotide sequences were subjected to comparative analysis regarding the amino acid sequence. The analysis has revealed that bk134P22.1 (GenBank Accession No. CAB56227) of 1296 bp, a human gene sequences, is a sequence homologous to that of the TSLC1 gene.

bk134P22.1 is a predicted gene based on the genomic sequence of a BAC clone 134P22 of 118951 bp (GenBank Accession No. AL035403).

cDNA library derived from human brain tissue was screened by use, as a probe, of an RT-PCR product having the sequence of bk134P22.1, which is expressed in human adult brain tissue, to thereby obtain seven distinct cDNA clones.

Through comparison of the nucleotide sequences of the clones, sequences of two full-length TSLL1 cDNA molecules which have different sequences in the 3′-end were obtained, the first one having a size of 2416 bp (PolyA was added to the end of the 2416th nucleotide within the 1st to 2416th nucleotides in the seuqence of SEQ ID NO: 2) and the second one having a size of 3557 bp (PolyA was added to the end of the 3557th nucleotide within the 1st to the 3557th nucleotides in the sequence of SEQ ID NO: 2), both molecules ended by two distinct selective polyadenylation sites originating from the identical gene.

The determined sequence of the present gene TSLL1 is shown in SEQ ID NO: 2. The amino acid sequence deduced therefrom is shown in SEQ ID NO: 1. This sequence corresponds to GenBank Accession No. AF363367. Although bk134P22.1 was predicted to contain an open reading frame (ORF) of 1296 bp, both of the two isoforms of TSLL1 cDNA that the inventors cloned contained a shorter ORF of 1194 bp encoding a protein of 398 amino acids, in which a sequence corresponding to the predicted exon 2 of the bk134P22.1 was not present.

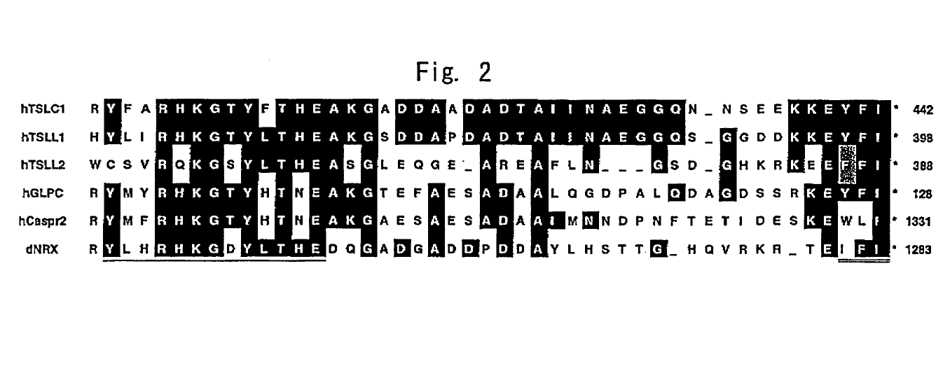

FIG. 2 shows amino acid sequence alignment among human TSLC1, human TSLL1, human TSLL2 (TSLL2 is a gene which is obtained from human adult brain-derived cDNA library through screening by use of F22162_{—}1 as a probe; TSLL2 cDNA of 2176 bp contains an ORF of 1164 bp encoding a protein of 388 amino acids) (GenBank Accession No. AF363368), human glycophorin C (hGLPC) (GenBank Accession No. P04921), human contactin-associated protein 2 (hCaspr2) (NP 054860), and Drosophia melanogaster Neurexin IV (dNRX) (X 86685) protein. Gaps are introduced in order to realize the best alignment. Conserved residues among the amino acid sequences are indicated by black boxes. The 13 amino acid residues which are essential for binding to protein 4.1 in hGLPC (Marfatia et al., 1995) are indicated by the underbar, whereas the three consensus amino acids at the COOH terminal for PDZ binding (Marfatia et al., 1997; Songyang et al., 1997) are indicated by the double underline.

The predicted amino acid sequence shows that TSLL1 encodes a membrane protein with an extracellular domain containing three immunogloblin-like C2-type loops with several potential N-glycosylation sites, a transmembrane domain, and a short cytoplasmic domain. As shown in FIG. 2, TSLC1 exhibits high homology to TSLL1 in the cytoplasmic domain (76% identical in the amino acid sequence). Moreover, TSLL1 show considerable similarity with TSLC1 in the entire amino acid sequence (37% identical in the amino acid sequence). The above results indicate that TSLL1 protein belongs to an immunoglubulin superfamily.

Determination of the Gene Structure

High-density filters of RPCI-11 human BAC library were purchased from Research Genetics and used according to the manufacture's recommendation. BAC DNA from clones 431A20 containing the TSLL1 gene were identified through hybridization by use of cDNA probes corresponding to the entire coding sequence of TSLL1. The gene structure of TSLL1 was determined by setting the respective cDNA sequences in the known genomic nucleotide sequence of BAC clone 134P22 (GenBank Accession No. AL035403). A portion of the genomic sequence of the TSLL1 gene was determined by use, as a template, of a BAC DNA clone from clones derived from a 431A20 clone.

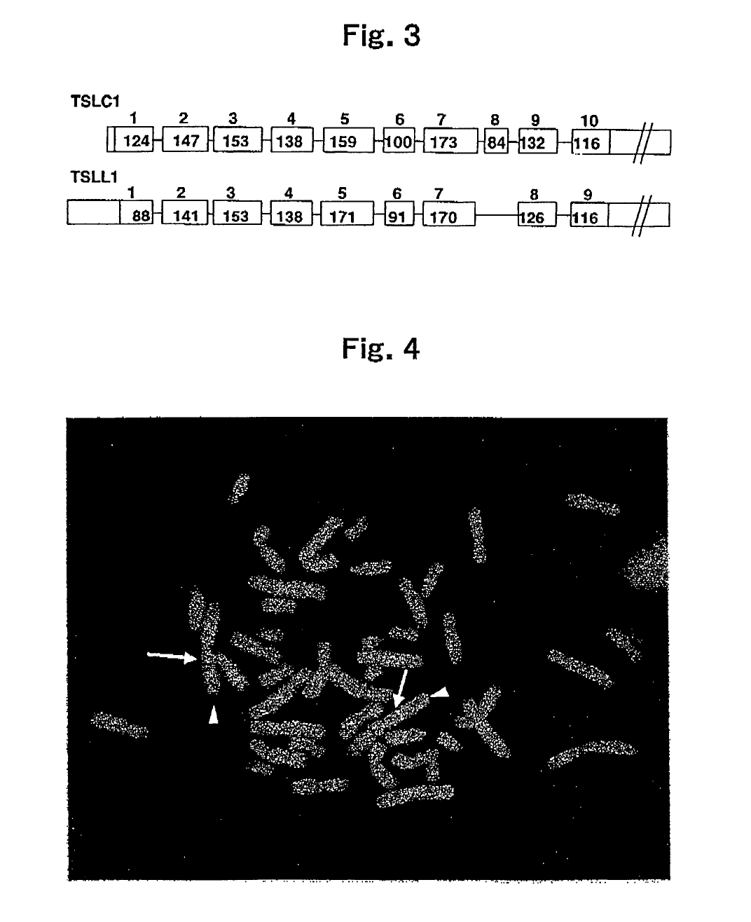

FIG. 3 shows a schematic representation of the human TSLL1 gene and the human TSLC1 gene. Each of the exons in the translation regions is surrounded by a square box in which the nucleotide length is shown, whereas exons in non-translation regions are left blank. The serial number of each exon is indicated above the corresponding square box.

The TSLL1 gene contains nine exons, and each of the exons spans a region of about 30 kb. Exon 1 is followed by seven relatively small exons which range from 91 to 171 bp in size, and a large, last exon 9 (FIG. 3). Exon 1 contains a start codon for methionine, and the codon is preceded by the Kozak's consensus sequence (Kozak, 1989).

The exon-intron structures are highly conserved between the TSLC1 gene and the TSLL1 gene except for exon 8 of the TSLC1 gene. Each gene has corresponding exons which are almost identical in size. The upstream regions of the TSLL1 gene are highly-rich in the content of guanine and cytosine residues and meet the criterion of CpG island of 754 bp (Antequera et al., 1993).

Fluorescence in situ Hybridization (FISH)

Fluorescence in situ hybridization (FISH) was performed in order to directly detect the chromosomal locus of the TSLL1 gene.

FISH was carried out by use of an established method (Pinkel et al., 1986). DNAs purified from BAC clones 431A20 were labeled with Spectrum Green-dUTP (Product of VYSIS) by use of a nick translation kit (Product of Boehringer) through the method which has been described previously (Shimura et al., 1999). The DNAs were used as probes for detection of the chromosomal locus of the TSLL1 gene. Probes for the telomeric region of human chromosome 1q were purchased from VYSIS and labeled with Spectrum Orenge-dUTP through the above-described method. After hybridization, cells and chromosomes were counterstained with 4′, 6-diamidino-2-phenylindole (DAPI) in an antifade solution (Product of Wako Pure Chemicals Industries, Ltd.) containing 0.1% p-phenylenediamine and 90% glycerol. Digital images were captured by use of a fluorescent microscope (Product of Olympus) and analyzed with Cyto Vision (Product of Applied Imaging). Through examination of at least 20 metaphase chromosomes, specific signals were detected in human chromosome 1q.

FIG. 4 shows the chromosomal locus of the TSLL1. Green signals (represented by triangles in FIG. 4) indicate DNAs containing TSLL1, whereas red signals (represented by arrows) indicate the telomeric regions in the long arm of chromosome 1.

As shown in FIG. 4, through hybridization of the BAC clones (431A20) with normal human metaphase chromosome, specific signals were detected on human chromosome 1q. Detection of this signal coincides with the finding that cDNA of TSLL1 has a sequence identical with a portion of the sequence of BAC clone 134P22 in chromosome 1q21.2-22.

Cell Lines

A human prostate cancer cell line PPC-1 was kindly provided by Dr. A. R. Brothman from the University of Utah. Human glioma cell lines (Hs683, SW1088, SW1783, DBTRG-05MG, CCF-STTG1, U87MG, U118MG, and U373MG) and human prostate cancer cell lines (PC3, LNCaP, and DU145) were obtained from American Type Culture Collection (ATCC). Human glioma cell lines (T98G, A172, YKG-1, and KG1-C) were obtained from the Health Science Research Resources Bank (Osaka). These cell lines were cultured according to the supplier's recommendation.

Northern Blotting Analysis

Human multiple-tissue Northern blot membrane, human adult brain poly(A)⁺ RNA, and a cDNA probe for human β-actin were purchased from Clontech. Poly(A)⁺ RNA of glioma or prostate cancer cells was extracted by use of a FastTrack 2.0 Kit (Product of Invitrogen). Poly(A)⁺ RNA (1 μg) was subjected to electrophoresis in a 1% agarose gel containing 2.1 M formamide and transferred onto a Hybond-N+ nylon membrane (Product of Amarsham). A coding sequence of TSLL1 gene was used as a probe. Hybridization was carried out at 42° C. for 18 hours in 5×SSC, 20 mM sodium phosphate, 1×Denhardt's solution, 0.2% SDS, 100 μg/ml denatured salmon testis DNA, 10% dextran sulfate, and 50% formamide. The membrane on which RNA had been transferred was then washed with 2×SSC/0.1% SDS at 42° C. for 15 minutes followed by 0.1×SSC/0.1% SDS at 50° C. for 15 minutes. Thereafter, the membrane was exposed to autoradiography for 1-4 days at −70° C. together with an intensifying screen.

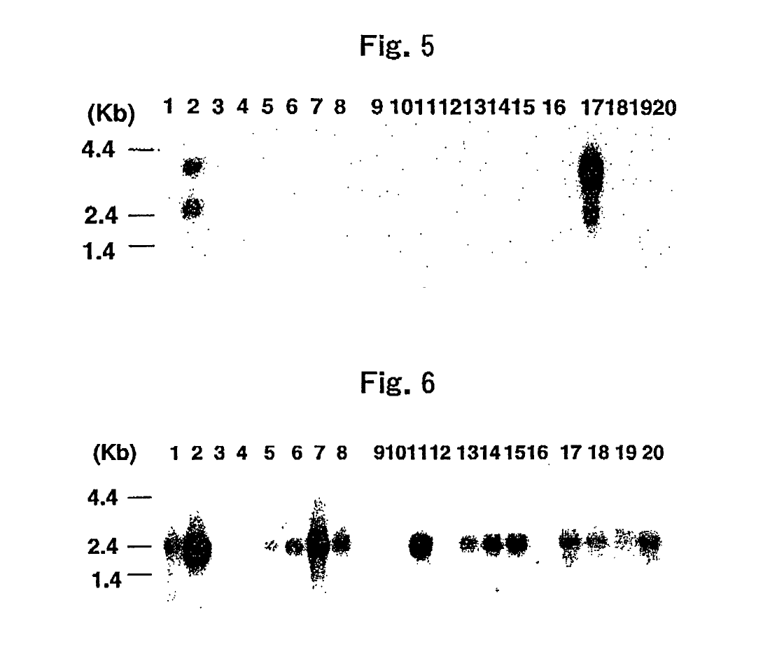

FIG. 5 shows the results of Northern blotting analysis of TSLL1 gene in 16 types of adult tissues and four types of fetal tissue. Each lane contained 2 μg of poly(A)⁺ RNA. Lane 1, heart; lane 2, brain; lane 3, placenta; lane 4, lung; lane 5, liver; lane 6, skeletal muscle; lane 7, kidney; lane 8, pancreas; lane 9, spleen; lane 10, thymus; lane 11, prostate; lane 12, testis; lane 13, ovary; lane 14, small intestine; lane 15, colon; lane 16, peripheral blood cell; lane 17, fetal brain; lane 18, fetal lung; lane 19, fetal liver; and lane 20, fetal kidney.

As shown in FIG. 5, the TSLL1 gene was expressed exclusively in human adult and fetal brains. Corresponding to the results of the cDNA clones, two distinct transcripts of 3.6 and 2.4 kb were present in almost the same amounts.

FIG. 6 shows the results of Northern blotting analysis of TSLL1 gene in 12 types of human glioma cell lines. β-actin is shown in the low position of each lane. Each lane contained 5 μg of poly(A)⁺ RNA. Lane 1, Hs683; lane 2, SW1088; lane 3, SW1783; lane 4, DBTRG-05MG; lane 5, CCF-STTG1; lane 6, U87MG; lane 7, U118MG; lane 8, U373MG; lane 9, T98G; lane 10, A172; lane 11, YKG-1; lane 12, KG1-C; and lane 13, adult brain.

The results of Northern blotting analysis shown in FIG. 6 indicate that TSLL1 expression was lost in all but a glioma cell line SW1783, suggesting that TSLL1 gene product functions to suppress the development or progression of human glioma.

From the above, it is clear that TSLL1 gene is a tumor suppressor gene.

Preparation of Antibody Against TSLL1

A portion of the amino acid sequence of TSLL1 protein (AEGGQSGGDDKKEYFI: SEQ ID NO: 7) was synthesized by means of a peptide synthesizer. The peptide was conjugated to a carrier protein (keyhole limpet hemocyanin; KLH) through a conventional method, to thereby prepare an immunogen. By use of the immunogen, a rabbit was immunized five times at intervals of 14 days, to thereby obtain immune serum. The immune serum was purified through a conventional method by use of a peptide affinity column which contained sulfolink-coupling gel to which synthesized peptide (C) (AEGGQSGGDDKKEYFI: SEQ ID NO: 7) had been bound, to thereby yield a polyclonal antibody (named AC2).

Accordingly, the aforementioned findings revealed that the antigen recognition site of AC2 has a sequence of AEGGQSGGODKKEYFI (SEQ ID NO: 7). This sequence is identical with the above-mentioned sequence of the synthesized peptide; thus, AC2 was found to be a polyclonal antibody specific to TSLL1 protein.

Detection of TSLL1 Protein by Use of the Antibody Against TSLL1

An expression vector for expressing TSLL1 was incorporated into cultured monkey-kidney-derived cells (COS-7), and then the cells in which TSLL1 had been expressed were isolated. The vectors were obtained in such a manner that DNA fragments yielded by PCR were integrated into a plasmid vector pcDNA 3.1 (product of Invitrogen). In the PCR procedure, cDNA prepared from human brain cell-derived poly(A)⁺ RNA was used as a template, and nucleotides having sequences of SEQ ID NOs: 3 and 4 were used as primers for amplification. A cell extract was subjected to Western blotting in accordance with a conventional method (Towbin et al., 1979, Burncutte et al., 1981). Cultured cells were collected, solubilized by use of a cell lysate solution containing 1% SDS, 1 mM EDTA, Hepes buffer (pH 7.5), and 100 μM Pefabloc and Complete (product of Boehringer Mannheim), and then centrifuged (10000×G, 10 minutes), to thereby yield a supernatant containing a protein. The protein (40 μg) was separated by means of SDS (10% to 20%) polyacrylamide gel electrophoresis, and transferred onto a polyvinyl lysine difluoride filter. A specific polyclonal antibody (AC2) against TSLL1 protein was added to the filter, and then reacted with the protein. The protein was detected by use of a donkey anti-rabbit IgG antibody which had been conjugated to alkaline phosphatase.

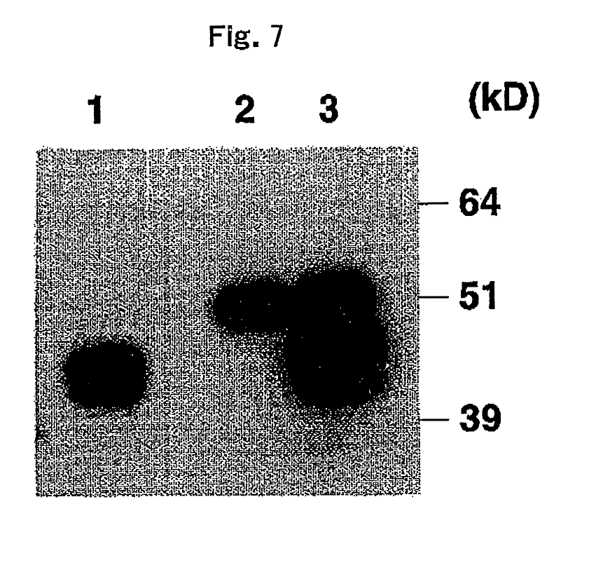

Through SDS polyacrylamide gel electrophoresis, the following three materials were isolated and transferred onto a membrane: entire cell extract of COS-7 cells which were caused to be expressed by introducing an expression vector for expressing TSLL1 (lane 1), a product obtained by immunoprecipitation of a cell extract of COS-7 cells in which an expression vector for expressing TSLL1 had not been introduced, with polyclonal antibody AC2 against TSLL1 (lane 2), and a product obtained through immunoprecipitation of entire cell extract of COS-7 cells in which an expression vector for expressing TSLL1 had been introduced, with polyclonal antibody AC2 against TSLL1 (lane 3). Subsequently, the respective products were hybridized through Western blotting by use of AC2 (FIG. 7). As a result, two TSLL1 proteins which had undergone post translational modification were detected with specificity. That is, proteins having molecular weights of about 40 kDa (lane 1) and about 45 kDa (lane 3) were detected. The signal corresponding to a molecular weight of about 50 kDa represents the heavy chain of IgG.

As described above, the present invention provides, among other things, a novel tumor suppressor gene, which enables provision of novel means useful in prevention, diagnosis, and treatment of cancers.

What is claimed is:

1. A protein having an amino acid sequence represented by SEQ ID NO: 1.

2. A gene comprising a nucleotide sequence encoding an amino acid sequence represented by SEQ ID NO: 1.

3. The gene as described in claim 2, which is a tumor suppressor gene.

4. A gene composed of a nucleotide sequence encoding an amino acid sequence represented by SEQ ID NO: 1.

5. The gene as described in claim 4, which is a tumor suppressor gene.

6. A gene comprising a nucleotide sequence represented by SEQ ID NO: 2.

7. The gene as described in claim 6, which is a tumor suppressor gene.

8. A gene composed of a nucleotide sequence represented by SEQ ID NO: 2.

9. The gene as described in claim 8, which is a tumor suppressor gene.

10. A gene expression vector harboring a gene as described in claim 2.

11. A transformant obtained through transformation with a gene expression vector as described in claim 10.

12. An antibody which is immunoreactive with a protein as described in claim 1, the entirety or a portion of the protein serving as an immunogen.


SEQUENCE LISTING (13 sequences)

SEQ ID NO: 1 - protein, 398 aa, Homo sapiens
MGAPAASLLLLLLLFACCWAPGGANLSQDDSQPWTSDETVVAGGTVVLKCQVKDHEDSSLQWSNPAQQTLYFGEKRALRDNRIQLVTSTPHELSISISNVALADEGEYTCSIFTMPVRTAKSLVTVLGIPQKPIITGYKSSLREKDTATLNCQSSGSKPAARLTWRKGDQELHGEPTRIQEDPNGKTFTVSSSVTFQVTREDDGASIVCSVNHESLKGADRSTSQRIEVLYTPTAMIRPDPPHPREGQKLLLHCEGRGNPVPQQYLWEKEGSVPPLKMTQESALIFPFLNKSDSGTYGCTATSNMGSYKAYYTLNVNDPSPVPSSSSTYHAIIGGIVAFIVFLLLIMLIFLGHYLIRHKGTYLTHEAKGSDDAPDADTAIINAEGGQSGGDDKKEYFI

SEQ ID NO: 2 - DNA, 3557 bp, Homo sapiens
  exon (141)..(1334)
ggtccccacctcggccccgggctccgaagcggctcgggggcgccctttcggtcaacatcgtagtccaccccctccccatccccagcccccggggattcaggctcgccagcgcccagccagggagccggccgggaagcgcgatgggggccccagccgcctcgctcctgctcctgctcctgctgttcgcctgctgctgggcgcccggcggggccaacctctcccaggacgacagccagccctggacatctgatgaaacagtggtggctggtggcaccgtggtgctcaagtgccaagtgaaagatcacgaggactcatccctgcaatggtctaaccctgctcagcagactctctactttggggagaagagagcccttcgagataatcgaattcagctggttacctctacgccccacgagctcagcatcagcatcagcaatgtggccctggcagacgagggcgagtacacctgctcaatcttcactatgcctgtgcgaactgccaagtccctcgtcactgtgctaggaattccacagaagcccatcatcactggttataaatcttcattacgggaaaaagacacagccaccctaaactgtcagtcttctgggagcaagcctgcagcccggctcacctggagaaagggtgaccaagaactccacggagaaccaacccgcatacaggaagatcccaatggtaaaaccttcactgtcagcagctcggtgacattccaggttacccgggaggatgatggggcgagcatcgtgtgctctgtgaaccatgaatctctaaagggagctgacagatccacctctcaacgcattgaagttttatacacaccaactgcgatgattaggccagaccctccccatcctcgtgagggccagaagctgttgctacactgtgagggtcgcggcaatccagtcccccagcagtacctatgggagaaggagggcagtgtgccacccctgaagatgacccaggagagtgccctgatcttccctttcctcaacaagagtgacagtggcacctacggctgcacagccaccagcaacatgggcagctacaaggcctactacaccctcaatgttaatgaccccagtccggtgccctcctcctccagcacctaccacgccatcatcggtgggatcgtggctttcattgtcttcctgctgctcatcatgctcatcttccttggccactacttgatccggcacaaaggaacctacctgacacatgaggcaaaaggctccgacgatgctccagacgcggacacggccatcatcaatgcagaaggcgggcagtcaggaggggacgacaagaaggaatatttcatctagaggcgcctgcccacttcctgcgccccccaggggccctgtggggactgctggggccgtcaccaacccggacttgtacagagcaaccgcagggccgcccctcccgcttgctccccagcccacccacccccctgtacagaatgtctgctttgggtgcggttttgtactcggtttggaatggggagggaggagggcggggggaggggagggttgccctcagccctttccgtggcttctctgcatttgggttattattatttttgtaacaatcccaaatcaaatctgtctccaggctggagaggcaggagccctggggtgagaaaagcaaaaaacaaacaaaaaacaaaaccctggagtgttaggaggagagtgaaggtagaggggtgaggaagggtaaggggcagggctggtttcagctgggggctctcaccagccctcctttcagcctctacaacagagcagcttcccagacttctccaggaacccagaaacgggatggttgtcggcaaaggttgggagtggcttttcctctggtagccacacacctgagcactacggacagggaggcaggtgccaccttgacacctctcttccatagcaatgggaaagtgatgagtgcgggagtcctgaggagatgtggcctgcagacaacatgcagccatgcagggacccaggactgtaacctggggaggacgcgggtccctgcaaggaagagtagatttggagaggaaggatggaggtggactctcaccccattccccccggaaatgaacaaagccgggccctttccataggaactgcccttggagatagcagagtgtggctgcccctccttgctccagcagcagtgggagaggcactgctctggggcctgaactgcctctgcttccccccctgaggggcccctcactcttacccaagactctggattgttgcacggcaaccactcctcccatggcattgctcagcaactacttctcccttcccggccaccctgtgcccccttcctggtcccaacgccagcccttcatccttcctccctcagcagccaggcagacataacaacaaaactactaaaaggagcttcactgcagtgagctgtttcctgcccaaactaagggaataatgtgaactgtgtgcatgtgtgtggtgtgtatgcatgtgtgcatgtgtgtgtgtgtgtgtgcatgtgtgtgagtgagtgagaggcagagcgaggaactgaggaggagggctaagagccaggggtcctgggcaagtggacagggctgtgggacatgttggggaggctttgggaatggggtattcctagtcagggttcacacctcacctgggatgttgttccatgctggtatttcctctgccacccccaatgcccatcggtcttggagaaaggagtccccgggtgtgtgtttgcccagctgtccattctatctctcccttaaacacagagcattcagcccttccctggatttccctcctctgagccatggagtcagtgccacagcctttgctatgcacctctcaggcctctccttggcgttgaccctggaaagacctaccaccacctattttttcccatagtctgtacccagtgagttgaaggctgggtccccacccttccttttgatttcctgtcttccttctcgtggccccagctggttgctgtggagatgaggttcctggtcctccctgtcctggctggactgccccgcctcagatccaggatgcccttggcatcgctcccaccctcccccagcttttcctccctggtctgacaatgggcatgcaaaaaggggcagctgcaatctagcaggcctgcccacccccttcagttcaggtaatacagttgtgaatcttccagccgctggttagggccttgggcaccacaggcagcccctcacctaagccggggcctactcctcttacaacagcaagagagccctggggccccaggcctgttgagcttcttgtctcccagcacccgcttttgggaaaatgacttttcctcttcaagctgaaccactctgtccatattacacagaagccatatttgtacgggggggtgggagggagaggggctgttgtgctgtgtgtgtctgtccaggggtgggggggtgggggaagggagcagggaggggaccgtgtatctttataatctttctaactctcctgtgctaatctcagaggggtcaccctcaatatatctggattatccgtgtc

SEQ ID NO: 3 - DNA, 30 bp, Artificial Sequence
  Gene Amplification Primer
cctttcggtcaacatcgtagtccaccccct

SEQ ID NO: 4 - DNA, 30 bp, Artificial Sequence
  Gene Amplification Primer
tacaagtccgggttggtgacggccccagca

SEQ ID NO: 5 - DNA, 18 bp, Artificial Sequence
  Gene Amplification Primer
acagccagccctggacat

SEQ ID NO: 6 - DNA, 23 bp, Artificial Sequence
  Gene Amplification Primer
ctagatgaaatattccttcttgt

SEQ ID NO: 7 - protein, 17 aa, Homo sapiens
CAEGGQSGGDDKKEYFI

SEQ ID NO: 8 - protein, 46 aa, Homo sapiens
RYFARHKGTYFTHEAKGADDAADADTAIINAEGGQNNSEEKKEYFI

SEQ ID NO: 9 - protein, 46 aa, Homo sapiens
HYLIRHKGTYLTHEAKGSDDAPDADTAIINAEGGQSGGDDKKEYFI

SEQ ID NO: 10 - protein, 42 aa, Homo sapiens
WCSVRQKGSYLTHEASGLEQGEAREAFLNGSDGHKRKEEFFI

SEQ ID NO: 11 - protein, 47 aa, Homo sapiens
RYMYRHKGTYHTNEAKGTEFAESADAALQGDPALQDAGDSSRKEYFI

SEQ ID NO: 12 - protein, 47 aa, Homo sapiens
RYMFRHKGTYHTNEAKGAESAESADAAIMNNDPNFTETIDESKEWLI

SEQ ID NO: 13 - protein, 45 aa, Drosophila melanogaster
RYLHRHKGDYLTHEDQGADGADDPDDAYLHSTTGHQVRKRTEIFI